(19) United States
(12) Patent Application Publication
(10) Pub. No.: US 2013/0071476 A1
(43) Pub. Date: Mar. 21, 2013

(54) Rapid Melt Controlled Release Taste-Masked Compositions

(71) Applicant: Subraman Rao Cherukuri, Vienna, VA (US)

(72) Inventor: Subraman Rao Cherukuri, Vienna, VA (US)

(21) Appl. No.: 13/589,101

(22) Filed: Aug. 31, 2012

Related U.S. Application Data
(60) Provisional application No. 61/525,270, filed on Aug. 19, 2011.

(51) Int. Cl. A61K 9/50; A61K 9/20; A61K 31/44; A61K 31/485; A61K 47/34

(52) U.S. Cl. CPC A61K 9/2081; A61K 9/5036; A61K 9/5042; A61K 9/5026; A61K 31/485; A61K 31/44
USPC 424/465; 424/484; 424/486; 424/488; 424/485; 424/464; 514/289; 514/357

(57) ABSTRACT

Rapid melt tablets that dissolve and release an active component in the oral cavity are comprised of a pharmaceutical active ingredient such as dextromethorphan complexed with a resin that is effective in taste-masking the otherwise bitter taste of the active making it convenient for oral administration. The drug/resin-complexed particles can be coated with water swellable or water insoluble polymers to impart controlled release properties to the active ingredient. A rapid melt tablet also comprises diluents, sweeteners, flavors, disintegrants and other excipients to form granules that can be compressed into tablets at low pressure without the need for a binding agent.

### CROSS-REFERENCE TO RELATED APPLICATIONS

This application claims the benefit of priority of U.S. Provisional Appln. No. 61/525, 270 filed on Aug. 18, 2011.

### FIELD OF THE INVENTION

The present invention relates generally to oral compositions that cover up or mask the unfavorable taste of the active ingredients so that they can be formulated into rapid melt tablets with a combined controlled release effect such that active pharmaceutical ingredients can be released over a long period of time and yet not cause an undesirable or bitter taste on the tongue, mouth and oral cavity.

### BACKGROUND OF THE INVENTION

Rapid melt tablets are a drug dosage form that has been available for a limited amount of over-the-counter (OTC) and prescription medications for some time. Rapid melts differ from traditional tablets in that they are designed to be dissolved on the tongue rather than swallowed whole. The rapid melt tablet serves as an alternative dosage form for patients who have difficulty in swallowing (dysphagia) or when compliance is a known issue and therefore an easier dosage form to take ensures that medication is taken. Common among all age groups, dysphagia is observed in about 35% of the general population, as well as up to 60% of the elderly institutionalized population and 18-22% of all patients in long-term care facilities. During the last decade, rapid melt tablets have become available in a variety of therapeutic markets, both OTC and by prescription. An additional reason to use a rapid melt tablet is the convenience of a tablet that can be taken without the need for water.

The difficulty in administration was experienced in particular by pediatrics and geriatrics patients, but this also applies to the patients who are ill in bed or traveling. Other groups that may experience problems using conventional oral dosage forms include the mentally ill, developmentally disable and patients who are uncooperative. A difficulty in swallowing (dysphasia) tablets or capsules is common problem among all age groups, especially in elderly and pediatrics. For this reasons, tablets that can dissolve or disintegrate in oral cavity, have attracted a great deal of attention. Many techniques have been reported for the formulation of fast dissolving tablets such as freeze drying/lyophilization, tablet moulding, spray drying, sublimation, direct compression, and mass extrusion.

Taste masking is one of the more critical functions in the preparation of the rapid melt tablet compositions because rapid melt tablets are solid dosage forms containing medicinal substances which disintegrate rapidly, usually in a matter of seconds, when placed on the tongue. As tablet disintegrates in mouth, the bitter taste of the drug is quickly perceived by the taste buds and other olefactory sense organs in the mouth resulting in a relatively unpleasant experience. Most drug and pharmaceutical actives are bitter in nature and effective taste masking formulations are always needed to hide the bitter taste in rapid melt compositions.

### Taste Masking Methodologies:

I. Coating Drug Particles with Inert Agents

Coating is an extremely useful technique for a number of applications in pharmaceutical field. Although it is used primarily for production of sustained release, gastro-intestinal dosage forms, it also has major applications in masking the unpleasant tastes. A great deal of attention has recently been focused on the usefulness of coated fine particles in pharmaceutical technology. By coating the drug particle with an appropriate polymer system, desirable properties can be imparted to the dosage form with the resultant elimination of undesirable properties such as mouthfeel and taste. Most non-toxic polymers that are insoluble at a pH of 7.4 and soluble at acidic pH would be acceptable for taste masking. Taste masking involves covering the total surface of the particle with enough coating so that the taste of the particle is not perceived by the users. As the particle size of drug gets smaller, the surface area to be coated grows exponentially, making it difficult to coat. The way in which taste-masked microcapsules are delivered is a very important parameter in selection of type of polymer coating for bitter drugs. The polymer coating should be such that taste masked microcapsules can withstand the hostile environment of preparation, storage (shelf life), handling, and finally consumption all the while ensuring that the drug is released at a specific time that is the most desirable for maximum drug bioavailability.

Drug particles can also be coated using spray congealing, microencapsulation, and fluidized bed coating. Of these, spray congealing and fluidized bed coating are the most effective taste masking techniques because they are cost effective, don't require a solvent, and can produce a more dense film than other methods. Another effective drug coating technique known in the art is micro-encapsulation, a process in which a relatively thin polymer coating is applied to small particles of the active solid. Coating agents include gelatin, povidone, hydroxypropyl methylcellulose (HPMC), ethyl cellulose, beeswax, carnauba wax, acrylics, and shellac. Bitter-tasting drugs can first be encapsulated to produce free-flowing microcapsules, which can then be blended with other excipients and compressed into tablets. Micro-encapsulation can be accomplished using a variety of methods including air suspension, coacervation, phase separation, spray drying and congealing, pan coating, solvent evaporation, and multi-orifice centrifugation.

### II. Inclusion Complexation

Inclusion complexation is a process in which the guest molecule is included in the cavity of a host. The complexing agent masks the bitter taste of the drug either by decreasing its oral solubility upon ingestion or by decreasing the number of drug particles exposed to taste buds, thereby reducing the perception of bitter taste.

Cyclodextrins are the most widely used complexing agent for inclusion complex formation. Cyclodextrins are cyclic oligosaccharides containing either six, seven, or eight glucose units, categorized accordingly as alpha, beta, and gamma cyclodextrin. Beta cyclodextrin is the most commonly used cyclodextrin for inclusion complexation because its cavity size fits in the aromatic ring present in most drug molecules. Physical forces such as van der Waal's forces and hydrophobic interactions stabilize the complex that is formed. The complex liberates the drug when diluted by a medium. In cases where the binary complex is not good enough to serve the purpose, ternary complexation, which involves the use of a third component in addition to the drug and cyclodextrin, is utilized for efficient taste masking. The third component is generally a low molecular weight hydrophilic polymer such as polyvinyl pyrolidone and HPMC

Iii. Melt Granulation:

Melt granulation is carried out at elevated temperatures (50-80° C.) using low-melting lipids like glyceryl behenate and glyceryl palmitostearate as disintegrants. This approach comes with its own drawbacks, however: It's not suitable for drugs with heat sensitivity, it runs the risk of negatively affecting the dissolution rate, and it can create stability issues due to the susceptibility of lipids to oxidative degradation.

### IV. Ion Exchange Resins:

The adsorption of bitter drugs onto synthetic ion exchange resins to achieve taste coverage has been well documented. Synthetic ion exchange resin was used in pharmacy and medicine for taste masking. Ion exchange resins are solid and suitably insoluble high molecular weight poly-electrolytes that can exchange their mobile ions of equal charge with the surrounding medium. The resulting ion exchange is reversible and stiochiometric, resulting in the displacement of one ionic species by another. Drugs with ionizable functionalities form ion exchange complexes with the resin; this complex (resinate) is insoluble and prevents the drug's exposure to the taste bud. On reaching the gastric lumen, the complex dissociates and releases the drug due to the exchange of drug with counter-ions. Because they are high molecular weight water-insoluble polymers, the resins are not absorbed by the body and are therefore inert.

The present invention relates generally to cough preparations which in the past have always been were formulated as liquids, syrups or suspensions. In view of the fact that there are a number of advantages in the use of solids over oral liquid dosage forms, the present invention deals with the formulation of tablet dosage form of a cough preparation combining the advantages of a rapid melt and controlled release within a single tablet. Moreover, solutions and suspensions are bulky and not generally convenient to carry around like that of a solid dosage form. They are also less microbiologically and chemically stable than their solid counterparts. The accuracy of oral liquid dose is also dependent on the patient measuring the dose carefully.

From a manufacturing point of view, solid oral dosage forms have more advantages over liquid oral dosage forms like they are more stable than liquids and possess longer expiration dates for easier shipping and handling requirements. Consequently, there is a need for less shelf space with no preservative requirements, and accurate dosages, i.e., a single dose administration is possible and readily achieved. Initially, most of the rapid melt, orally dispersible tablets were formulated having immediate release and therapeutic action. Later during the further development of rapid release formulations, in addition to meeting the patient's needs and also in order to decrease the frequency of dosage needed, the controlled release (or extended release) orally disintegrating tablet was developed. The ability to achieve good patient compliance is the one of the major advantage of this type of dosage form. It is easy to administer to pediatric as well as geriatric patients as in addition to achieving fast melting of tablet and consequent release of the active in the mouth, there is the ability to develop excellent control the release of drug.

U.S. Pat. No. 6,024,981 to Khankari et. al., discloses a rapidly dissolving robust dosage form directed to a hard tablet that can be packaged, stored and processed in bulk. The solid tablet dissolves in the mouth of a patient with a minimum of grit. The tablet contains an active ingredient mixed into a matrix of a non-direct compression filler and a relatively high lubricant content.

U.S. Pat. No. 5,989,583 to Amselem, discloses a dry solid lipid composition suitable as an oral dosage form. The composition contains a lipophilic substance, at least one fat which is a solid at about 25.degree. C. and at least one phospholipid present in an amount of about 2 to 40% by weight of the composition. However, the resultant product is a dry solid lipid composition.

U.K. Patent Application. GB 2,195,892,892 to Amselem discloses pharmaceutical chewable tablets with improved palatability. The lipid-containing tablets include a lipid material having a melting point from about 26° C. to about 37° C., a particulate dispersant material, an emulsifier and a safe and effective amount of a pharmaceutically active material. The tablets of the lipid composition exhibit improved palatability, and effective dispersion in the mouth and stomach. pharmaceutical chewable tablets with improved palatability. composition exhibit improved palatability, and effective dispersion in the mouth and stomach.

U.S. Pat. No. 5,837,285 to Nakamichi et. al., discloses fast soluble tablets that can be produced by a simple method. The tablet base is a sugar alcohol. The mixture of the sugar alcohol and a drug is subjected to compressive shaping prior to drying in the process. The dry solid tablet can be produced by modification of conventional tabletting technology and possesses physico-chemical stability.

U.S. Pat. No. 5,753,255 to Chavkin et. al. discloses a chewable medicinal tablet. The tablet contains about 30 to about 95% by weight of a capric triglyceride and a medicinally active ingredient up to 60% by weight. If the medicinally active ingredient is less than about 30% by weight, then the composition also contains up to 10% by weight of a member of the group consisting of glyceryl monostearate, a mixture of glyceryl monostearate and glyceryl monopalmitate, and a mixture of glyceryl monostearate and glyceryl distearate.

U.S. Pat. No. 5,320,848 to Geyer et. al. discloses a non-aqueous chewable composition for oral delivery of unpalatable drugs. The drug is intimately dispersed or dissolved in a pharmaceutically-acceptable lipid that is solid at room temperatures. The lipid material desirably readily melts with the application of mild temperatures, i.e. about 55 to 95 C.

U.S. Pat. No. 4,937,076 to Lapidus, discloses a chewable aspirin and buffering material tablet in a single dosage form. The buffering materials are integrally dispersed and bound in a fatty material of chocolate, synthetic chocolate or hydrogenated tallow. The fatty material individually coats the aspirin and buffering material.

U.S. Pat. No. 4,684,534 to Valentine discloses quick-liquefying, chewable tablets. The tablets have a harder outer shell which inhibits penetration of liquid, and a softer interior which quickly liquefies when the tablet and shell are broken into pieces and contacted by the liquid. The excipient or base material of the tablet is made from carbohydrates held together with small quantities of a carbohydrate disintegrant such as maltodextrin. The tablets can contain active ingredients such as pharmaceuticals, breath sweeteners, vitamins and dietary supplements.

U.S. Pat. No. 4,609,543 to Morris et. Al. discloses a soft homogeneous antacid tablet. The tablet contains solid antacid particles thoroughly coated with a mixture composed of a fatty material or oil, a surfactant, and a flavor. The fat or oil is present in an amount of from about 25% to about 45% of the mixture. The primary particle size of the antacid is less than 100 millimicrons (mμ)

U.S. Pat. No. 4,446,135 to Valentine, discloses chewable calcium carbonate-containing antacid tablets having good mouth feel properties. The good mouth feel properties of the tablet are obtained by using calcium carbonate of a particular particle size in combination with certain excipients. The calcium carbonate is present in an effective amount and has a size from about 5 to 50 microns in diameter.

U.S. Pat. No. 5,320,848 to Geyer et. al. discloses a non-aqueous chewable composition for oral delivery of unpalatable drugs. The drug is intimately dispersed or dissolved in a pharmaceutically-acceptable lipid that is solid at room temperatures. The lipid material desirably readily melts with the application of mild temperature, i.e., from about 75° to about 95° C.

U.S. Pat. No. 5,837,285 to Nakamichi et. al. discloses fast soluble tablets that can be produced by a simple method. The tablet base is a sugar alcohol. The mixture of the sugar alcohol and a drug is subjected to compressive shaping prior to drying in the process. The dry solid tablet can be produced by modification of conventional tableting technology and possesses physico-chemical stability.

U.S. Pat. No. 4,446,135 to Fontaine, discloses chewable calcium carbonate-containing antacid tablets having good mouth feel properties. The good mouth feel properties of the tablet are obtained by using calcium carbonate of a particular particle size in combination with certain excipients. The calcium carbonate is present in an effective amount and has a size from about 5 to 50 microns in diameter.

U.S. Pat. No. 4,327,077 to Puglia et al. discloses a compressed chewable antacid tablet which has good flexibility, is breakage resistant and disintegrates immediately upon chewing. The tablet is formed of a re-crystallized fatty material, such as chocolate, a bulking material and an active ingredient bound up in the particles of the re-crystallized fatty material. The preferred re-crystallized fatty material is a chocolate or a synthetic chocolate.

U.S. Pat. No. 4,327,076, to Puglia et al., also discloses a compressed chewable antacid tablet which has good flexibility, is breakage resistant and disintegrates immediately upon chewing. The tablet is formed of particles of the antacid or other active ingredients which are admixed with particles formed of edible fat or oil absorbed on a fat-absorbing material, such as microcrystalline cellulose. Upon chewing, the tablet is quickly converted to a smooth creamy non-gritty palatable emulsion.

U.S. Pat. No. 5,320,848 to Geyer et. al. discloses a non-aqueous chewable composition for oral delivery of unpalatable drugs. The drug is intimately dispersed or dissolved in a pharmaceutically-acceptable lipid that is solid at room temperatures. The lipid material desirably readily melts with the application of mild temperatures, i.e. about 55 to 95 C.

U.S. Published Patent Appln. No. 2006/0115529 to Jeong describes and claims fast-melting tablets having taste-masking and sustained release properties comprising particles of an active ingredient and ion-exchange resin complex that masks unpleasant tastes associated with the active ingredient. The resin complex particles can be coated or uncoated to impart sustained release properties to the active ingredient. A fast-melting tablet also comprises a dry disintegrant and bulk diluent to form highly plastic granules that are subsequently compressed into tablets.

However, the prior art compositions contain various disadvantages. For example, some tablet formulations may be incompletely chewed due to the poor palatability of the composition. Such compositions may also have a gummy texture, and are subject to “taste fatigue,” i.e., the composition is perceived to be less palatable after ingestion of multiple doses. Further, the disintegrants and other materials used in such chewable tablets may prevent rapid and effective delivery of active materials to the stomach.

There is a need for a rapid-melt, composition that has a lubricious, palatable mouth-feel that behaves like a liquid when consumed and yet acts like a solid in many other ways. The need encompasses those compositions in which little to substantially no biting or chewing is necessary in order to cause the composition to melt, disintegrate, decompose, or otherwise break down or apart in the mouth. Such compositions are ideal for uses in the fields of pediatric and geriatric care, that is, for use with people or domestic mammals that do not have any teeth. These compositions are particularly useful for pediatric, geriatric patients or for those with limited ability to swallow traditional dosage forms.

Controlled-release pharmaceutical compositions are well known and described in the prior art. These generally comprise (a) at least one pharmaceutically active substance and one or more polymers, hydrating agents, dissolution agents, bulking agents, encapsulating agents and the like. Suitable controlled release polymers generally comprise a first polymer component and one or more secondary polymer components having different dissolution and drug wettability and release-ability characteristics from that of the first polymer. Known polymers useful in drug delivery systems may be soluble, insoluble, permeable, impermeable or biodegradable according to the physiological environment they are in. These may also be natural or synthetic polymers or copolymers.

### BRIEF DESCRIPTION OF THE DRAWINGS

FIG. 1 is a plot of the in-vitro drug release (cumulative percent drug release) of the Dextromethorphan HBr from the Rapid Melt Controlled Release Tablets in 0.1 N HCl (Example-15)

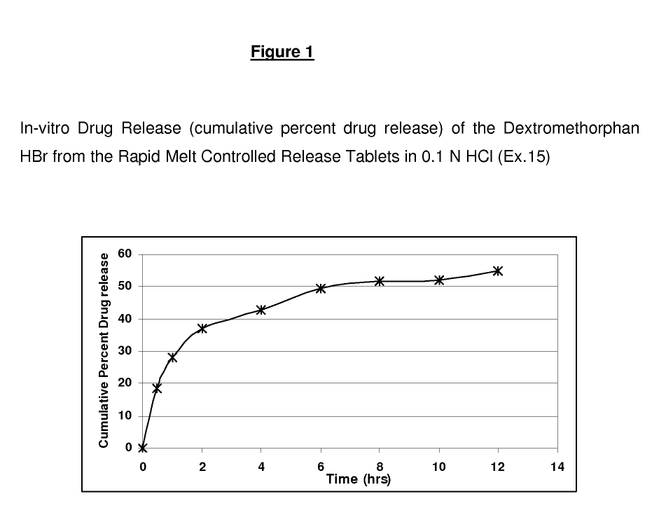

FIG. 2 is a plot of the in-vitro drug release (cumulative percent drug release) of the Dextromethorphan HBr from the rapid melt controlled release tablets in 1.2 pH followed by 6.8 pH phosphate buffer (Example-15)

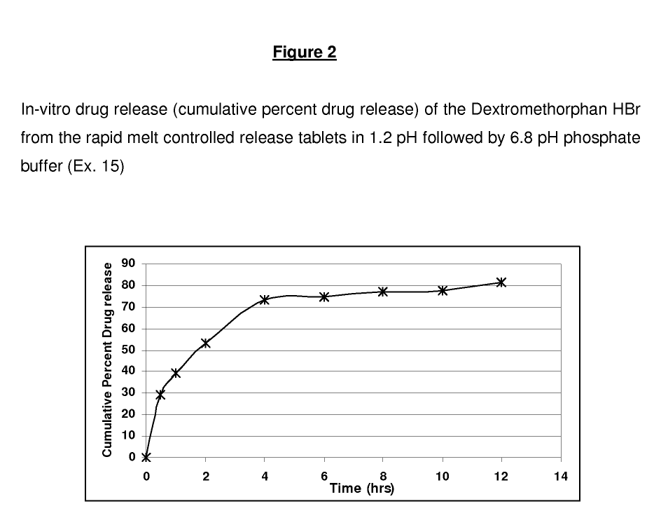

### SUMMARY OF THE INVENTION

The process of the present invention comprises the encapsulation of an active ingredient such as a cough suppressant or expectorant that has unpleasant taste or odor. In addition to masking the taste or odor, the compositions of the present invention extend the release of active ingredient by coating it with a control release polymer without the need for a binder component. More specifically, in the present invention the tablet releases the drug in a controlled way by disintegrating rapidly in the mouth. This provides combined advantages of both rapid melt and controlled release formulations. In addition to the advantages of rapid melt tablets known in the art, the present invention provides the ability to vary the controlled release in a single tablet. When used with drugs with a short half-life, controlled release can mean less frequent dosing and thus better compliance thereby reduce variations in plasma/blood levels for more consistent result.

### DETAILED DESCRIPTION OF THE INVENTION

The present invention is direction to rapid melt pharmaceutical formulations that provide a uniform drug release of the taste masked drug. For taste masking purposes, the method is particle coating of the active ingredient so as to result in a good mouth feel with no grittiness during dissolution of the active in the mouth. As discussed supra, there are many techniques which can mask the unpleasant taste and odor of active such as the drug-resin complex. But there may a problem in this type of taste masking due to the poor compressibility due to the use of resins. To avoid this problem, the coated multi-particulates of the drug-resin complex are not agglomerated during compression when a minimum compression force is applied. This also extends the controlled the release of drug.

Ion exchange resins are cross-linked; water insoluble, polymer-carrying, ionizable functional groups. Drugs can be loaded onto the resins by an exchanging reaction, and hence, a drug-resin complex (drug resinate) is formed. Ion exchange is a reversible process in which ions of like sign are exchanged between a liquid and a solid which has a highly insoluble body in contact with it. The drug is released from the resinates through ion exchange in the gastrointestinal fluid, followed by drug diffusion. Since the resins are high molecular weight water insoluble polymers, they are not absorbed by the body and are therefore inert. Drug molecules attached to the resins are released by appropriate charged ions in the gastrointestinal tract, followed by diffusion of free drug molecules out of the resins as shown below,

Resin−Drug++X+--------------->Resin− . . . X++Drug+ (1)

Resin+Drug−++X−----------------->Resin+ . . . X−+Drug (2)

Where, X and Y are ions in the gastrointestinal tract

Strong acid cation resins (sulfonated styrene-divinyl benzene copolymer products) can be used to mask the taste of basic drugs having a bitter taste, as they function through out the entire pH range. Weak acid cation exchange resins function at the pH values above 6. Similarly, strong base anion exchange resin function throughout the entire pH range, while the weak base anion exchange resins function well below pH 7.

The ion exchange phenomenon as mentioned previously is driven by electrostatic interactions between the resin and oppositely charged drugs. The interactions are strongly governed by the pH of the medium (at a certain pH, one of the two entities may become neutralized depending on their pKa, thus eliminating the charge), or by the presence of competing ions (for example, presence of high ionic strength buffers may reduce the electrostatic interactions between the resin and drug due to a shielding/competitive binding effect. The reversibility of this interaction is exploited in oral drug delivery, in which the resin may carry the drug and release the payload in a certain region of the gastrointestinal tract due to a pH change or presence of competing ions. Naturally, the prerequisite for applicability of ion exchange resin for a drug is the presence of charged groups on the drug. Examples of drugs that have used ion exchange resins in formulation include dextromethorphan, diclofenac, ibuprofen, and paroxetine.

There are four major types of ion exchange resins available today. Strongly acidic cation exchange resins with sulfonic acid functionality e.g. Dowex 50, Amberlite-120 and Sodium polystyrene sulfonate. weakly acidic cation exchange resins with carboxylic functionality e.g. Amberlite IPR-88, Amberlite IPR-64 & IPR-64M and Amberlite IRC-50; strongly basic anion exchange resins with quaternary ammonium functionality e.g. Dowex-1, Amberlite IR400. weakly basic anion exchange resins with secondary and tertiary amine functionality e.g. Amberlite IR 4B, Dowex 2, polyamine methylene resin. Strong acid resins are so named because their chemical behavior is similar to that of a strong acid. The resins are highly ionized in both the acid (R—SO₃H) and salt (R—SO₃Na) form.

Dextromethorphan (DXM or DM) is an anti-tussive or cough suppressant drug. It is one of the active ingredients in many over-the-counter cold and cough medicines, such as Robitussin, NyQuil, Dimetapp, Vicks, Coricidin, Tussin, Delsym, and others, including generic labels. Dextromethorphan has also found other uses in medicine, ranging from pain relief to psychological applications. It is sold in syrup, tablet, spray, and lozenge forms. In its pure form, dextromethorphan occurs as a white powder. Dextromethorphan is used to temporarily relieve coughs caused by the common cold, the flu, or other conditions. Dextromethorphan will relieve a cough but will not treat the cause of the cough or speed recovery. It works by decreasing activity in the part of the brain that causes coughing.

Dextromethorphan HBr can also be formulated in combination with an anti-histamine (such as diphenylpyraline hydrochloride), and/or a decongestant (such as phenylephrine hydrochloride). This combination medication is used to treat symptoms caused by the common cold, flu, allergies, hay fever, or other breathing illnesses (e.g., sinusitis, bronchitis). Dextromethorphan is a cough suppressant that affects a certain part of the brain (cough center), reducing the urge to cough. Decongestants help relieve stuffy nose symptoms while. Anti-histamines relieve watery eyes, itchy eyes/nose/throat, runny nose, and sneezing.

The IUPAC name of Dextromethorphan is (+)-3-methoxy-17-methyl-(9α,13α,14α)-morphine. It is the dextrorotatory enantiomer of the methyl ether of levorphanol which is an opioid analgesic. The generally available salt form of dextromethorphan is dextromethorphan monohydrated hydro-bromide. Like other anti-tussives, it also exhibits pharmacological activity on the cough center located in the medulla oblongata area of the brain. Dextromethorphan also has other actions in the brain, which may help it work to treat PBA (Pseudobulbar affect). It works by blocking certain receptors, which prevent the action of excitatory brain chemicals. The average dose necessary for effective anti-tussive therapy is 20-45 mg in adults.

Anti-histamines inhibit the action of histamine by blocking it from attaching to histamine receptors. Generally the term “anti-histamine” refers only to H1 antagonists, also known as H-1 anti-histamines. By blocking histamine in the capillaries. anti-histamines can reduce the intensity of allergic symptoms. Anti-histamines cross the blood-brain barrier (BBB) and antagonize the H-1 receptors centrally. Its effect on central H-1 receptors cause drowsiness.

In one aspect of the invention, dextromethorphan HBr is first encapsulated in a resin such Amberlite IRP 69. Other suitable resins useful in the encapsulation procedure include Amberlite IPR-88, Amberlite IPR-64 & IPR-64M and Amberlite IRC-50; strongly basic anion exchange resins with quaternary ammonium functionality e.g. Dowex-1, Amberlite IR400. weakly basic anion exchange resins with secondary and tertiary amine functionality e.g. Amberlite IR 4B, Dowex 2, polyamine methylene resin.

The present invention then comprises a rapid melt composition for the relief of coughs and chest congestion comprising one or more bitter-tasting actives that is first incorporated into a drug-resin ion-exchange complex that is then micro-encapsulated within a carrier selected from the group consisting of polymers, biopolymers, fats, waxes, gums and mixtures thereof which is further incorporated within a fast dissolving polymer matrix. The active is preferably selected from the group consisting of anti-tussives, expectorants, antihistamines and mixtures thereof. More specifically, the active is selected from the group consisting of dextromethorphan, diclofenac, ibuprofen, phenylephrine, doxylamine, imenhydrinate, brompheniramine, chlorpheniramine and paroxetine brompheniramine maleate, chlorpheniramine maleate, phenylepherine hydrochloride, pseudoephedrine and mixtures thereof.

The active is encapsulated within a drug-resin complex wherein the resin is selected from the group consisting of acidic cation exchange resins with sulfonic acid functionality, weakly acidic cation exchange resins with carboxylic functionality, strongly basic anion exchange resins with quaternary ammonium functionality, weakly basic anion exchange resins with secondary and tertiary amine functionality, polyamine methylene resins and mixtures thereof. The drug-resin complex is then further encapsulated with water-swellable, hydrophilic or water insoluble polymers selected from the group consisting of polyvinylpyrrolidine, hydroxyl-propylcellulose, hydroxylpropylmethyl cellulose methyl cellulose, hydroxyethyl cellulose vinyl acetate copolymers, polysaccharides, polyethyleneoxide, methacrylic acid copolymers, maleic anhydride/methyl vinyl ether copolymers and natural gums or pharmaceutically acceptable hydrophobic polymers like ethylcellulose, glyceryl behenate and glyceryl palmito behenate. Surprisingly, the formulations of the present invention are achieved without the necessity of incorporating additional binder compounds to maintain cohesion as are necessary in the rapid melt compositions of the prior art.

Preferably the rapid melt tablet consists of a bitter-tasting active which is administered in an amount of from about 30 mg. to about 60 mg per 500 mg of the composition. The composition is further formulated as a flash-bead, dissolvable gel, liquid emulsion or a compressed tablet.

The compositions of the present invention may further comprise a diluent/bulking material selected from the group consisting of mannitol, dextrate, sorbitol, glycerol and mixtures thereof. Other suitable components include salivating agents, emulsifiers such as sodium lauryl sulfate and polysorbate 80. The composition may also comprise a disintegrant such as polyethylene glycol, diluent/bulking materials such as magnesium stearate, flavors, stabilizers, sweeteners and the like.

The encapsulated dextromethorphan HBr can then be formulated as a tablet dosage form, flash beads, soft chews, a liquid. suspension, controlled release tablets or a gel form. When the tablet form is produced a lubricant tabletting agent such as

The rapid-melt compositions of the present inventive subject matter are preferably anhydrous, that is, they do not contain any water. The lack of water in the inventive compositions allows high doses of active materials or combinations of active materials to be incorporated into the compositions due to the stability of the active materials in the absence of the water. It is contemplated, however, that the compositions may optionally include an amount of water. The amount of water present will depend on the active ingredients to be delivered, but generally will be present in an amount less than 2.0% by weight of the composition. Preferably, the water will be present in an amount less than 1.0% by weight of the composition.

The rapid-melt compositions of the present inventive subject matter contain at least one or more additional disintegrants, bulking agents and/or excipients. These are ingredients useful in keeping the composition in its state, may be either solid or liquid, and may include, without limitation, a high melting point fat or waxy material such as lipid materials, polyethylene glycols (PEG), waxes and other fats. Preferably, the compositions of the present invention contain a mixture of these agents. The solid disintegrants useful in the compositions of the present invention have a melting point of about 25 to 90° C., and preferably about 37° C. When more than one disintegrant is used in the present invention, the melting point of the combination of the disintegrants will remain within the range of 25 to 90° C. and preferably about 37° C. The inventive subject matter contemplates the use of mixtures of solid disintegrants and liquid disintegrants. For a non-limiting example, the present inventive subject matter contemplates mixing a small amount of a high-melting point lipid with a liquid disintegrant to achieve a disintegrant that attains the desired product characteristics. These characteristics include such factors as mouth feel, rapidity of melting in the mouth, appearance, flavor and compatibility with active materials and therapeutic active materials.

Among the lipid materials useful in the compositions of the present inventive subject matter are those which are commercially available and commonly used in confectionery and other food products. Such lipid materials include, without limitation, cocoa butter, hydrogenated tallow, hydrogenated vegetable oils, hydrogenated cotton seed oil, palm kernel oil, soybean oil, stannol esters, and derivatives and mixtures thereof. Hydrogenated vegetable oils (such as hydrogenated palm kernel oil), cocoa butter, and cocoa butter substitutes are among the preferred useful lipid materials. Additional disintegrants may include glycerol esters, polyalcohol esters, polyoxyethylelne esters of hydrophilic and hydrophobic balances from 0.5 to above 20 and polyethylene glycols. Other examples include saccharides such as monosaccharides and oligosaccharides. Examples of monosaccahrides include: dextrose, dextrose monohydrate, lactose, mannose, fructose, etc. Liquid disintegrants may also be used. Examples of liquid disintegrants are, without limitation, polysaccharides, gum solutions, water, corn syrup, hydrogenated starch hydrolates, glycerin, polypropylene glycol, and mixtures thereof. It should be noted that these ingredients, when used may be present in quantities to not affect the constituency of the product so that the final product retains a predominantly solid constituency. In some aspects, the liquid disintegrants may not exceed about 5% of the composition.

The amount of disintegrant present in the rapid-melt composition of the present inventive subject matter is from about 0.01% to about 70% by weight of the final composition. Preferably, the amount of disintegrant is from about 0.01% to about 50% by weight of the composition. More preferably the disintegrant is present from about 15% to about 30% by weight of the composition.

The disintegrant is used to provide good melt away properties to the composition while preventing a gritty texture being imparted by the composition. The disintegrant aids in the fast melting of the composition when placed in the mouth of a user.

The rapid-melt composition of the present invention also contains a salivating agent. As is used herein, “salivating agent” means a material that promotes greater salivation in the user of the compositions of the present inventive subject matter. The salivating agent helps create salivation in the mouth of the mammal using the inventive compositions. This is an important feature since the present compositions are intended to be taken by the patient without the aid of water to help in the transporting of the composition to the stomach of the patient. The salivating agent can be, without limitation, an emulsifier or a food acid that initiates salivation in the mouth of the patient.

Examples of emulsifiers useful as salivating agents in the compositions of the present inventive subject matter include, without limitation, alkyl aryl sulfonates, alkyl sulfates, sulfonated amides and amines, sulfated and sulfonated esters and ethers, alkyl sulfonates, polyethoxylated esters, mono-, di-, and triglycerides, diacetyl tartaric esters of monoglycerides, polyglycerol esters, sorbitan esters and ethoxylates, lactylated esters, phospholipids such as lecithin, polyoxyethylene sorbitan esters, proplyene glycol esters, sucrose esters, and mixtures thereof. The emulsifier may be either saturated or unsaturated. It should be noted that some of the emulsifiers that are salivating agents may also function as disintegrants.

Examples of food acids useful as salivating agents in the inventive compositions include, without limitation, citric acid, malic acid, tartarate, food salts such as sodium chloride and salt substitutes, potassium chloride, and mixtures thereof.

The amount of salivating agent present in the rapid-melt composition of the present inventive subject matter is from about 0.05% to about 15% by weight of the final composition. Preferably, the amount of salivating agent from about 0.3% to 0.4% by weight of the composition.

Keeping the amount of salivating agent present in the inventive composition within these limits for weight percentage is important to enhance the desirable properties of the compositions. More particularly, the low amount of salivating agent present in the compositions aid in the compositions retaining the physical state and the rapidity of melting in the mouth of a mammal.

The rapid-melt compositions of the present inventive subject matter further contain a diluent/bulking material. The use of a diluent/bulking material is necessary to serve as a free-flow imparting agent which aids in the moisturizing of the composition when chewed, that is, the diluent/bulking material aids in the process-ability of the compositions. The diluent/bulking material also serves to reduce the concentration of the active materials and add bulk to the composition. Examples of diluent/bulking materials useful in the compositions of the present inventive subject matter include, without limitation, silicon dioxide, sugars, starches, lactose, sucrose, sorbitol, fructose, talc, erythitol, xylitol, mannitol, maltitol, isomalt, dextrose, maltose, lactose, microcrystalline celluloses and mixtures thereof. It should be noted that some of the diluents/bulking materials may also function as disintegrants.

The amount of diluent/bulking material present in the rapid-melt compositions is from about 0.5% to about 99% by weight of the final composition. Preferably, the amount of diluent/bulking material is from about 2.0% to about 95% by weight of the final composition.

The rapid-melt compositions of the present inventive subject matter may optionally contain a further slipping agent to aid in the palatability of the composition after it melts in the mouth of the mammal. The slipping agent may be a further lipid material, as is described above for disintegrants, or another material which aids in the “slipping” of the composition through the mouth and down the esophagus of the mammal.

The compositions of the present invention may be compressed into tablets or made into granules, beads or particles for direct consumption/administration. The granules, beads or particles may be further processed into additional dosage forms such as tablets, capsules, caplets or suspensions and emulsions.

As discussed above, the preferably anhydrous nature of the present inventive compositions allows for very high doses of active materials to be incorporated therein. The amount of active material present in the inventive compositions will vary depending on the particular active used, but generally will be present in an amount of about 0.001% to 70% by weight of the composition. Preferably, the active ingredients used in the inventive compositions are prophylactic or therapeutic active ingredients. Prophylactic or therapeutic active materials which can be used in the present invention are varied. A non-limiting list of such materials includes the following: antitussives, antihistamines, decongestants, alkaloids, mineral supplements, vitamins, antacids, analgesics, antibiotics, cough suppressants, mucolytics, and mixtures thereof.

The following examples are provided to more specifically set forth and define the process of the present invention. It is recognized that changes may be made to the specific parameters and ranges disclosed herein and that there are a number of different ways known in the art to change the disclosed variables. And whereas it is understood that only the preferred embodiments of these elements are disclosed herein as set forth in the specification and drawings, the invention should not be so limited and should be construed in terms of the spirit and scope of the claims that follow herein.

### EXAMPLES

### Example 1

### Dextromethorphan HBr/Sodium polystyrene sulfonate Resin Complex (Ratio of Drug and Resin 50:50)

| Material Name | Amount (g) |
|---|---|
| Dextromethorphan HBr USP | 50.00 |
| Sodium polystyrene sulfonate USP/NF | 50.00 |
| Purified water | 1000 ml |
| Total | 100.00 |

### Process:

The Dextromethorphan-sodium polystyrene sulfonate complex is prepared by, sifted the 50 gyms. of sodium polystyrene sulfonate particle size in between 106-125 micrometers (μm) and 50 gyms. of #30 (ASTM) sieved Dextromethorphan HBr were premixed then the premixed blend is dispersed in 1000 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example 2

### Dextromethorphan HBr/Sodium polystyrene sulfonate Resin Complex (Ratio of Drug and Resin 40:60)

| Material Name | Amount (g) |
|---|---|
| Dextromethorphan HBr USP | 40.00 |
| Sodium polystyrene sulfonate USP/NF | 60.00 |
| Purified water | 1500 ml |
| Total | 100.00 |

### Process:

The Dextromethorphan-sodium polystyrene sulfonate complex is prepared by, sifted the 60 gyms. of sodium polystyrene sulfonate particle size in between 106-125 micrometers (μm) and 40 gyms. of #30 (ASTM) sieved Dextromethorphan HBr were premixed then the premixed blend is dispersed in 1500 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example 3

### Dextromethorphan HBr/Hydrogen polystyrene sulfonate Resin Complex (Ratio of Drug and Resin 50:50)

| Material Name | Amount (g) |
|---|---|
| Dextromethorphan HBr USP | 50.00 |
| Hydrogen polystyrene sulfonate | 50.00 |
| Purified water | 1000 ml |
| Total | 100.00 |

### Process:

The Dextromethorphan-hydrogen polystyrene sulfonate complex is prepared by, sifted the 50 gyms. of hydrogen polystyrene sulfonate particle size in between 106-125 micrometers (μm) and 50 gyms. of #30 (ASTM) sieved Dextromethorphan HBr were premixed then the premixed blend is dispersed in 1000 ml of purified water USP and subjected to stirring using REMI-mechanical stirrer for 12 hrs at a stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example 4

### Dextromethorphan HBr/Hydrogen polystyrene sulfonate Resin Complex (Ratio of Drug and Resin 40:60)

| Material Name | Amount (g) |
|---|---|
| Dextromethorphan HBr USP | 40.00 |
| Hydrogen polystyrene sulfonate | 60.00 |
| Purified water | 1500 ml |
| Total | 100.00 |

### Process:

The dextromethorphan-hydrogen polystyrene sulfonate complex is prepared by, sifted the 60 gyms. of hydrogen polystyrene sulfonate particle size in between 106-125 micrometers (μm) and 40 gyms. of #30 (ASTM) sieved Dextromethorphan HBr were premixed then the premixed blend is dispersed in 1500 ml of purified water USP and subjected to stirring using REMI-mechanical stirrer for 12 hrs at a Stirrer RPM of 1300-1400 at room temperature. Then the drug-resin complex separated from the supernatant, another 1000 mls of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example 5

### Dextromethorphan HBr/Polacrilex Resin Complex (Ratio of Drug and Resin 50:50)

| Material Name | Amount (g) |
|---|---|
| Dextromethorphan HBr USP | 50.00 |
| Polacrilex resin | 50.00 |
| Purified water | 1000 ml |
| Total | 100.00 |

### Process:

The Dextromethorphan-Polacrilex resin complex is prepared by, sifted the 50 gms of Polacrilex resin particle size in between 106-125 micrometers (μm) and 50 Gyms. of #30 (ASTM) sieved Dextromethorphan HBr were premixed then the premixed blend is dispersed in 1000 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example 6

### Dextromethorphan HBr/Polacrilex Resin Complex (Ratio of Drug and Resin 40:60)

| Material Name | Amount (g) |
|---|---|
| Dextromethorphan HBr USP | 40.00 |
| Polacrilex resin | 60.00 |
| Purified water | 1500 ml |
| Total | 100.00 |

### Process:

The Dextromethorphan-Polacrilex resin complex is prepared by, sifted the 60 gyms. of Polacrilex resin particle size in between 106-125 micrometers (μm) and 40 Gyms. of #30 (ASTM) sieved Dextromethorphan HBr were premixed then the premixed blend is dispersed in 1500 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106-170 μm.

### Example 7

### Dextromethorphan HBr/Polacriline potassium Resin Complex (Ratio of Drug and Resin 50:50)

| Material Name | Amount (g) |
|---|---|
| Dextromethorphan HBr USP | 50.00 |
| Polacriline potassium | 50.00 |
| Purified water | 1000ml |
| Total | 100.00 |

### Process:

The Dextromethorphan-Polacriline potassium complex is prepared by, sifted the 50 gms. of polacriline potassium particle size in between 106-125 micrometers (μm) and 50 gms. of #30 (ASTM) sieved Dextromethorphan HBr were premixed then the premixed blend is dispersed in 1000 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example 8

### Dextromethorphan HBr/Polacriline potassium Resin Complex (Ratio of Drug and Resin 40:60)

| Material Name | Amount (g) |
|---|---|
| Dextromethorphan HBr USP | 40.00 |
| Polacriline potassium | 60.00 |
| Purified water | 1500 ml |
| Total | 100.00 |

### Process:

The Dextromethorphan-Polacriline potassium complex is prepared by, sifted the 60 gms. of Polacriline potassium particle size in between 106-125 micrometers (μm) and 40 gyms. of #30 (ASTM) sieved Dextromethorphan HBr were premixed then the premixed blend is dispersed in 1500 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

### Example-9

### Dextromethorphan HBr/Polacriline potassium Resin Complex (Ratio of Drug and Resin 30:70)

| Material Name | Amount (g) |
|---|---|
| Dextromethorphan HBr USP | 30.00 |
| Polacriline potassium | 70.00 |
| Purified water | 2000 ml |
| Total | 100.00 |

### Process:

The Dextromethorphan-Polacriline potassium complex is prepared by, sifted the 70 Gyms. of Polacriline potassium particle size in between 106-125 micrometers (μm) and 30 Gyms. of #30 (ASTM) sieved Dextromethorphan HBr were premixed then the premixed blend is dispersed in 2000 ml of purified water USP and subjected to stirring using REMI-mechanical stirrer for 12 hrs at a Stirrer RPM of 1300-1400 at room temperature. Then the drug-resin complex separated from the supernatant, another 1000 ml of the fresh de-ionized water was added and stirred again for 4 hrs at room temperature, filter the resultant product and filtrate drug-resin complex was dried in an oven at 50° C.-55° C. till the moisture content reaches to 5-7% measured using MB45 moisture analyzer. Sift the dried drug-resin complex and the final particle size should be in between 106 μm-170 μm.

Cumulative % Drug Release

| Time (hrs) | Example-2 | Example-4 | Example-6 | Example-8 |
|---|---|---|---|---|
| 0.5 | 34.68 | 37.25 | 41.26 | 40.25 |
| 1 | 51.25 | 54.69 | 43.52 | 42.69 |
| 2 | 62.65 | 57.99 | 52.36 | 48.69 |
| 4 | 64.66 | 61.25 | 59.69 | 65.66 |
| 6 | 64.72 | 61.26 | 60.99 | 65.71 |
| 8 | 64.89 | 61.65 | 64.36 | 65.85 |
| 10 | 64.89 | 61.66 | 68.96 | 65.99 |
| 12 | 65.00 | 62.98 | 69.69 | 66.00 |

### Examples 10 to 12

### Examples for Polymer coated Drug-Resin Complex by Fluid Bed Coating

| | Example10 | Example11 | Example12 |
|---|---|---|---|
| Material Name | Amount (g) | Amount (g) | Amount (g) |
| Dextromethorphan HBr-Resin | 934.50 | 948.00 | 922.00 |
| complex | | | |
| Ethyl cellulose USP | 50.40 | — | — |
| Methacrylic acid co-polymer | — | 60.00 | — |
| NF | | | |
| Hypromellose USP/Ph. Eur | — | — | 40.00 |
| Purified Talc USP | 5.00 | 6.00 | 4.00 |
| Tri-ethyl citrate NF | — | 12.00 | — |
| Propylene Glycol USP/EP | 10.10 | — | — |
| Polyethylene glycol 3350 NF | — | — | 8.00 |
| Isopropyl alcohol* | 820.00 | 975.00 | — |
| Purified water USP* | — | — | 650.00 |

Note:
*Indicates the solvent removed by drying during process

### Process:

Step-01: Polymer dissolved in about 70-80% of total Isopropyl alcohol or water under continuous stirring using a lab stirrer at an rpm of 300-500.
Step-02: Purified talc sieved through #120(ASTM) sieve and homogenized using 10-20% of Isopropyl alcohol or water for 10 min. using a homogenizer running at an RPM of 500-700.
Step-03: Plasticizer added to the above step-02 solution under continuous stirring.
Step-04: Drug-resin complex (From Example 02) whose particle size is in between 106-170 μm charged to Fluid Bed product bowl and coating solution sprayed using a top spray coating technique, maintaining Inlet temperature in between 50-55° C. and product temperature in between 45-50° C.
Step-05: After completion of coating, the dried granules were sifted so as the final particle size should be in between 125-250 μm.
In-Vitro Drug Release (Cumulative Percent Drug Release) of the Dextromethorphan HBr from the Polymer Coated Drug-Resin Complexes in 0.1 N HCl.

Drug release studies were carried out using USP-27 dissolution apparatus (Type II, paddle). The formulated tablet was added to 500 ml of 0.1 N HCl. The temperature of the medium was maintained at 37° C.±0.5° C. and was stirred at 50 rpm. Sample (10 ml) withdrawn at predetermined time interval over a period of 12 hrs. After each sampling, equal amount of the medium was added. The sample withdrawn was filtered to remove particulate matter and drug content was analyzed by HPLC system at a wavelength of 280 nm.

| | Cumulative % Drug Release | | |
|---|---|---|---|
| Time (hrs) | Example-10 | Example-11 | Example-12 |
| 0.5 | 19.20 | 20.34 | 30.25 |
| 1 | 28.43 | 32.20 | 43.52 |
| 2 | 38.20 | 40.23 | 52.36 |
| 4 | 43.21 | 46.65 | 59.69 |
| 6 | 50.11 | 54.65 | 64.76 |
| 8 | 50.55 | 57.89 | 67.55 |
| 10 | 51.66 | 61.89 | 67.56 |
| 12 | 53.25 | 62.26 | 67.56 |

### Examples 13 to 14

### Examples for Polymer encapsulation of Drug-Resin Complex by Granulation Method

| | Example-13 | Example-14 |
|---|---|---|
| Material Name | Amount (g) | Amount (g) |
| Dextromethorphan HBr-Resin complex | 250.00 | 230.00 |
| Ethyl cellulose USP | 50.00 | — |
| Hypromellose USP/Ph. Eur | — | 70.00 |
| Isopropyl alcohol* | 150.00 | — |
| Purified water USP* | — | 210.00 |

Note:
*Indicates to solvent removed by drying during process

### Process:

Step-01: The polymer was dissolved in isopropyl alcohol or water under continuous stirring using a lab stirrer at an rpm of 300-500.
Step-02: The drug-resin complex (From Example-02) whose particle size is in between 106-170 μm was granulated using high shear granulator maintaining impeller RPM in between 150-175, chopper RPM between 1200-1500.
Step-03: The above wet granules are dried in hot air oven maintained the temperature in between 50-55° C. till the moisture content of granules reaches 5-7%. Moisture content checked in MB45 Moisture analyzer at 100° C.
Step-05: The dried granules are sifted so as the final particle size should be in between 125-250 μm.
In-Vitro Drug Release (Cumulative Percent Drug Release) of the Dextromethorphan HBr from the Polymer Coated Drug-Resin Complexes in 0.1 N HCl.

Drug release studies were carried out using USP-27 dissolution apparatus (Type II, paddle). The formulated tablet was added to 500 ml of 0.1 N HCl. The temperature of the medium was maintained at 37° C.±0.5° C. and was stirred at 50 rpm. Sample (10 ml) withdrawn at predetermined time interval over a period of 12 hrs. After each sampling, equal amount of the medium was added. The sample withdrawn was filtered to remove particulate matter and drug content was analyzed by HPLC system at a wavelength of 280 nm.

| Time (hrs) | Example-13 | Example-14 |
|---|---|---|
| 0.5 | 20.21 | 23.14 |
| 1 | 26.13 | 30.20 |
| 2 | 37.26 | 41.15 |
| 4 | 44.21 | 46.25 |
| 6 | 50.21 | 53.15 |
| 8 | 51.15 | 56.39 |
| 10 | 51.26 | 58.96 |
| 12 | 53.55 | 62.11 |

### Example 15

### Rapid Melt Controlled Release Tablets Using Fluid Bed Coated Drug-Resin Complex Granules

The following formula for controlled release rapid melt tablets containing 30 mg of Dextromethorphan HBr in each tablet. Coated drug-resin complex particles are from example-10, whose particle size should be 125 μm-250 μm.

| Material Name | mg/tablet |
|---|---|
| Polymer encapsulated drug-resin complex | 100.78 |
| Mannitol Granules USP/NF | 329.06 |
| Mannitol powder USP | 67.50 |
| Microcrystalline-Cellulose Powder NF/Ph. Eur. | 135.00 |
| Sucralose powder NF | 3.38 |
| Natural orange Flavor | 3.38 |
| Crospovidone USP | 27.00 |
| Polysorbate 80 NF | 0.68 |
| FD&C Yellow #6 | 1.69 |
| Magnesium Stearate NF | 5.20 |
| Silicon Dioxide NF | 0.88 |
| Purified talc USP | 0.47 |
| Total | 675.00 mg/tablet |

### Process:

Step 1: Sift the Polymer encapsulated drug-resin complex granules (Example-10) whose particle size in between 125 μm-250 μm through #40 sieve.
Step 2: Add #30 sieved ¾ parts of Mannitol granules, microcrystalline cellulose, and Mannitol powder to active.

### Step 3: Adsorb the Polysorbate 80 on Crospovidone.

Step 4: Mix remaining ¼ parts of Mannitol granules, sucralose, orange flavor and step-3 mixture then sieved through #40 sieve.
Step 5: Mix Step 2 and step 4 mixtures and sieved through #40 sieve.
Step 6: Add #50 sieved FD&C yellow #6 to step 5 mixtures and blended for 10 mins using double cone blender at an RPM of 6-8.
Step 7: Sift Magnesium Stearate, talc and silicon dioxide through #50 sieve into a plastic container lined with poly bag, add to step 6 blended mixture and blend for 3-4 min using double cone blender at an RPM of 6-8 then compress blend part into tablet using ten stations pilot press tableting machine at an average hardness of 6 Kg.

These compressed tablets disintegrated in 25-30 seconds as tested in five volunteers with no observed bitter taste during disintegrate and after swallowed.

In-Vitro Drug Release (Cumulative Percent Drug Release) of the Dextromethorphan HBr from the Rapid Melt Controlled Release Tablets (Example-15) in 0.1 N HCl.

Drug release studies were carried out using USP-27 dissolution apparatus (Type II, paddle). The formulated tablet was added to 500 ml of 0.1 N HCl. The temperature of the medium was maintained at 37° C.±0.5° C. and was stirred at 50 rpm. Sample (10 ml) withdrawn at predetermined time interval over a period of 12 hrs. After each sampling, equal amount of the medium was added. The sample withdrawn was filtered to remove particulate matter and drug content was analyzed by HPLC system at a wavelength of 280 nm.

| | Cumulative percent drug release |
|---|---|
| Time (hrs) | (Example-15) |
| 0.5 | 18.51 |
| 1 | 28.23 |
| 2 | 37.10 |
| 4 | 42.91 |
| 6 | 49.54 |
| 8 | 51.85 |
| 10 | 52.00 |
| 12 | 54.77 |

In-Vitro Drug Release (Cumulative Percent Drug Release) of the Dextromethorphan HBr from the Rapid Melt Controlled Release Tablets (Example-15) in 1.2 pH Followed by 6.8 pH phosphate Buffer.

Drug release studies were carried out using USP dissolution apparatus (Type II, paddle). The formulated tablet was added to 500 ml of pH 1.2 for the first two hour and 900 ml of Phosphate buffer (pH 6.8) for the remaining ten hours. The temperature of the medium was maintained at 37° C.±0.5° C. and was stirred at 50 rpm. Sample (10 ml) withdrawn at predetermined time interval over a period of 12 hrs. After each sampling, equal amount of the medium was added. The sample withdrawn was filtered to remove particulate matter and drug content was analyzed by HPLC system at a wavelength of 280 nm.

| | Cumulative percent drug release |
|---|---|
| Time (hrs) | (Example-15) |
| 0.5 | 29.40 |
| 1 | 39.27 |
| 2 | 49.21 |
| 4 | 73.16 |
| 6 | 74.55 |
| 8 | 77.26 |
| 10 | 77.43 |
| 12 | 81.30 |

### Example 16

### Rapid Melt Controlled Release Tablets Using Fluid Bed Coated Drug-Resin Complex Granules

The following formula for controlled release rapid melt tablets containing 60 mg of Dextromethorphan HBr in each tablet. Coated drug-resin complex particles are from example-10, whose particle size should be 125 μm-250 μm.

| Material Name | mg/tablet |
|---|---|
| Polymer encapsulated drug-resin complex | 201.60 |
| Mannitol Granules USP/NF | 229.61 |
| Mannitol powder USP | 67.50 |
| Microcrystalline-Cellulose Powder NF/Ph. Eur. | 135.00 |
| Sucralose powder NF | 3.38 |
| Natural orange Flavor | 3.38 |
| Sodium starch Glycolate NF | 27.00 |
| Sodium Lauryl sulfate NF | 0.68 |
| FD&C Yellow #6 | 1.69 |
| Stearic acid NF | 5.20 |
| Total | 675.00 mg/tablet |

### Process:

Step 1: Sift the Polymer encapsulated drug-resin complex granules (Example-10) whose particle size in between 125 μm-250 μm through #40 sieve.
Step 2: Add #30 sieved ¾ parts of Mannitol granules, microcrystalline cellulose, and mannitol powder to active.
Step 3: Adsorb the Polysorbate 80 on sodium starch glycolate.
Step 4: Mix remaining ¼ parts of Mannitol granules, sucralose, orange flavor and step-3 mixture then sieved through #40 sieve.
Step 5: Mix Step 2 and step 4 mixtures and sieved through #40 sieve.
Step 6: Add #50 sieved FD&C yellow #6 to step 5 mixtures and blended for 10 mins. using double cone blender at an RPM of 6-8.
Step 7: Sift stearic acid through #50 sieve into a plastic container lined with poly bag, add to step 6 blended mixture and blend for 3-4 min using double cone blender at an RPM of 6-8 then compress blend part into tablet using ten stations pilot press tableting machine at an average hardness of 6 Kg.

These compressed tablets disintegrated in 25-30 seconds as tested in five volunteers with out observed bitter taste during disintegrate and after swallowed.

### Example 17

### Rapid Melt Controlled Release Flash Beads Using Fluid Bed Coated Drug-Resin Complex (with Fat)

The following formula for controlled release rapid melts flash beads containing 30 mg of Dextromethorphan HBr in each dose.

| Material Name | mg/dose |
|---|---|
| Polymer encapsulated drug-resin complex | 100.78 |
| Sugar 6X GRASS | 355.20 |
| Bakers Special Sugar NF | 350.00 |
| Sucralose Powder NF | 5.00 |
| Crospovidone USP | 30.00 |
| Cocoa Butter GRASS | 80.00 |
| Sorbitan Monostearate | 30.00 |
| Polyethylene glycol 3350 | 30.00 |
| Sodium Lauryl Sulfate NF | 0.50 |
| Polysorbate 80 NF | 1.50 |
| Natural orange Flavor | 10.00 |
| FD&C Yellow #6 | 2.00 |
| Silicon dioxide NF | 5.00 |
| Total | 1000.00 |

### Process:

1. Deodorized Cocoa butter and polyethylene glycol 3350 granular were melted in a bowl using a hot plate. Temperature and time for melting were 38-40° C. and 5-7 min. Temperature should not exceed above 50° C.-70° C. during process. Towards end of melting of above materials, Sodium lauryl sulfate, Polysorbate-80K and Sorbitan Monostearate were added and temperature recorded at 65-70° C. It should not exceed 70° C.-80° C.
2. Sugar 6×, Bakers special sugar, Sucralose, flavor and color were sieved through #30 sieve to a plastic tub lined with a poly bag. Now this blend was transferred to a double cone blender and to this Crospovidone (previously sieved through #30ASTM sieve). Blended for 5 min. at an RPM of 6-9.
3. Encapsulated active (previously sieved through #30 sieve) (From Example-10) whose particle size is in between 125-250 μm and flavor (previously sieved through #40 sieve) were added to above Step-02 material in blender and continue blending process for 5 min. at an RPM of 6-8. Transfer the blended material to a plastic tub lined with a poly bag.
4. The blend part was added to step-01 melted material under continuous mixing and mixed well. Temperature was recorded as 55-60° C.
5. This well mixed material was cooled till the temperature of reaches in between 25 to 30° C.
6. This cooled material was Extruder in a single screw extruder using 0.5 mm screen, maintained the RPM at 1200-1300 and extrudes was collected to a SS tray. Temperature of extrudes was 28-30° C.
7. The extrudes are spheronized in a spheronizer using 1 mm checker plate, maintained the rpm at 1100-1500 and spherical beads were collected to SS tray.
8. Colloidal Silicon dioxide sieved through #52 sieve and add to the above finished Product.
9. The mouth dispersion time was 10-12 sec. and no bitter or unacceptable taste was observed. There was no aftertaste was observed when the beads are completely melted.

### Example 18

### Rapid Melt Controlled Release Flash Beads Using Fluid Bed Coated Drug-Resin Complex (without Fat)

The following formula for controlled release rapid melts flash beads containing 30 mg of Dextromethorphan HBr in each dose.

| Material Name Active | mg/dose |
|---|---|
| Polymer encapsulated drug-resin complex | 100.80 |
| Sugar 6X GRASS | 355.62 |
| Bakers Special Sugar NF | 450.00 |
| Sucralose Powder NF | 5.00 |
| Crospovidone USP | 30.00 |
| Polyethylene glycol 3350 | 30.00 |
| Sodium Lauryl Sulfate NF | 0.50 |
| Polysorbate 80 NF | 1.50 |
| Natural orange Flavor | 10.00 |
| FD&C Yellow #6 | 2.00 |
| Silicon dioxide NF | 5.00 |
| Total | 1000.00 |

### Process:

1. Polyethylene glycol 3350 granular was melted in a bowl using a hot plate. Temperature and time for melting were 38-40° C. and 5-7 min. Temperature should not exceed above 50° C.-70° C. during process. Towards end of melting of above materials, Sodium lauryl sulfate and Polysorbate-80K were added and temperature recorded at 65-70° C. It should not exceed 70° C.-80° C.
2. Sugar 6×, Bakers special sugar, Sucralose, flavor and color were sieved through #30 sieve to a plastic tub lined with a poly bag. Now this blend was transferred to a double cone blender and to this Crospovidone (previously sieved through #30ASTM sieve). Blended for 5 min. at an RPM of 6-9.
3. Encapsulated active (previously sieved through #30 sieve) (From Example-10) whose particle size is in between 125-250 μm and flavor (previously sieved through #40 sieve) were added to above Step-02 material in blender and continue blending process for 5 min. at an RPM of 6-8. Transfer the blended material to a plastic tub lined with a poly bag.
4. The blend part was added to step-01 melted material under continuous mixing and mixed well. Temperature was recorded as 55-60° C.
5. This well mixed material was cooled till the temperature of reaches in between 25 to 30° C.
6. This cooled material was Extruder in a single screw extruder using 0.5 mm screen, maintained the RPM at 1200-1300 and extrudes was collected to a SS tray. Temperature of extrudes was 28-30° C.
7. The extrudes are spheronized in a spheronizer using 1 mm checker plate, maintained rpm at 1100-1500 and spherical beads were collected to SS tray.
8 Colloidal Silicon dioxide sieved through #52 sieve and added to the above finished product.
9. The mouth dispersion time was 10-12 sec. and no bitter or unacceptable taste was observed. There was no aftertaste was observed when the beads are completely melted.

### Example 19

### Rapid Melt Controlled Release Flash Beads Using Fluid Bed Coated Drug-Resin Complex (with Out Fat)

The following formula for controlled release rapid melts flash beads containing 60 mg of Dextromethorphan HBr in each dose.

| Material Name Active | mg/dose |
|---|---|
| Polymer encapsulated drug-resin complex | 201.60 |
| Sugar 6X GRASS | 254.82 |
| Bakers Special Sugar NF | 450.00 |
| Sucralose Powder NF | 5.00 |
| Crospovidone USP | 30.00 |
| Polyethylene glycol 3350 | 30.00 |
| Sodium Lauryl Sulfate NF | 0.50 |
| Polysorbate 80 NF | 1.50 |
| Natural orange Flavor | 10.00 |
| FD&C Yellow #6 | 2.00 |
| Silicon dioxide NF | 5.00 |
| Total | 1000.00 |

Process: Process is same as formulae-18.

### Example 20

### Soft Chews Using Fluid Bed Coated Drug-Resin Complex with Sugar

The following formula for soft chews containing 30 mg of Dextromethorphan HBr in each tablet. Coated drug-resin complex particles are from example-10, whose particle size should be 125 μm-250 μm.

| Material Name | mg/chew |
|---|---|
| Polymer encapsulated drug-resin complex | 100.80 |
| Mono- and Diglycerides | 129.79 |
| Distilled Monoglyceride (Panalite 90DK) | 64.89 |
| PEG 3350 Granular NF | 86.52 |
| Partially Hydrogenated Soybean and Cotton seed oil | 21.63 |
| Acetylated Monoglyceride | 108.15 |
| Maltodextrin | 369.67 |
| Bakers Special Granular Sugar | 616.48 |
| Maltitol Syrup NF | 432.62 |
| Natural Orange Flavor Powder | 21.63 |
| Micronised Sucralose Powder NF | 8.11 |
| HPMC K100LV USP | 10.82 |
| Concentrated Color Dispersion Red | 2.16 |
| Magnesium Stearate NF | 32.45 |
| Purified Talc USP | 21.63 |
| Colloidal Silicon Dioxide USP | 64.89 |
| Natural Orange Flavor Powder | 64.89 |
| Micronised Sucralose Powder NF | 2.70 |
| Concentrated Color Dispersion Red | 3.24 |

### Process:

Step-01: Sift the Polymer encapsulated drug-resin complex granules (Example-10) whose particle size in between 125 μm-250 μm, Hypromellose, Maltodextrin, Baker's Special Sugar, and Sucralose through #40 sieve (ASTM).
Step-02: Add Mono- and Triglycerides, Distilled Monoglyceride, PEG 3350 Granular NF, Partially Hydrogenated Soybean and Cotton seed oil, Acetylated Monoglyceride to the Groan Kettle; allow the mixture to melt.
Step-03: Add Maltitol Syrup 80/55 and Molten mixture from step-02 to BPG mixer. Mix in forward direction.
Step-04: Add sifted material from step-01 to the BPG mixture at temperature of approximately 105° F.-140° F. Close the lid. Mix in forward direction.
Step-05: Add #50 (ASTM) sieved Orange Flavor into BPG mixture. Mix in forward direction.
Step-06: Unload the material from BPG mixture and fill into plastic totes as per required dose. These soft chews tested in five volunteers no bitter taste observed during and after chewing.

### Example-21

### Soft Chews Using Fluid Bed Coated Drug-Resin Complex with Out Sugar

The following formula for soft chews containing 30 mg of Dextromethorphan HBr in each tablet. Coated drug-resin complex particles are from example-10, whose particle size should be 125 μm-250 μm.

| Material Name | mg/chew |
|---|---|
| Polymer encapsulated drug-resin complex | 100.80 |
| Mono and Diglycerides | 129.79 |
| Acetylated Monoglyceride | 108.15 |
| Coconut Oil | 86.52 |
| Sorbitol Powder | 1126.75 |
| Maltodextrin | 216.31 |
| Maltitol Syrup NF | 173.05 |
| Liquid Orange Flavor | 10.82 |
| Natural Orange Flavor Powder | 10.82 |
| Micronized Sucralose Powder NF | 8.11 |
| Concentrated Color Dispersion Red | 2.16 |
| Magnesium Stearate NF | 32.45 |
| Purified Talc USP | 21.63 |
| Colloidal Silicon Dioxide USP | 64.89 |
| Natural Orange Flavor Powder | 64.89 |
| Micronized Sucralose Powder NF | 2.70 |
| Concentrated Color Dispersion Red | 3.24 |

### Process:

Step 1: Sift the Polymer encapsulated drug-resin complex granules (Example-10) whose particle size in between 125 μm-250 μm, Maltodextrin, Sorbitol Powder and Sucralose through #40 sieve (ASTM).
Step 2: Add Mono and Diglycerides, Acetylated Monoglyceride and coconut to the Groen Kettle; allow the mixture to melt.
Step 3: Add Maltitol Syrup 80/55 and Molten mixture from step-02 to BPG mixer. Mix in forward direction.
Step 4: Add sifted material from step-01 to the BPG mixer at a temperature of approximately 105° F.-140° F. Close the lid. Mix in forward direction.
Step 5: Add Orange Flavor (Both liquid and powder) into BPG mixer. Mix in forward direction.
Step 6: Unload the material from BPG mixer and fill into plastic totes as per required dose. These soft chews tested in five volunteers no bitter taste observed during and after chewing.

### Example 22

### Soft Chews Using Fluid Bed Coated Drug-Resin Complex with Out Sugar

The following formula for soft chews containing 60 mg of Dextromethorphan HBr in each tablet. Coated drug-resin complex particles are from example-10, whose particle size should be 125 μm-250

| Material Name | mg/chew |
|---|---|
| Polymer encapsulated drug-resin complex | 201.60 |
| Mono and Diglycerides | 129.79 |
| Acetylated Monoglyceride | 108.15 |
| Coconut Oil | 86.52 |
| Crystalline Maltitol | 1025.95 |
| Maltodextrin | 216.31 |
| Maltitol Syrup NF | 173.05 |
| Liquid Orange Flavor | 10.82 |
| Natural Orange Flavor Powder | 10.82 |
| Micronized Sucralose Powder NF | 8.11 |
| Concentrated Color Dispersion Red | 2.16 |
| Magnesium Stearate NF | 32.45 |
| Purified Talc USP | 21.63 |
| Colloidal Silicon Dioxide USP | 64.89 |
| Natural Orange Flavor Powder | 64.89 |
| Micronized Sucralose Powder NF | 2.70 |
| Concentrated Color Dispersion Red | 3.24 |

### Process:

Step-01: Sift the Polymer encapsulated drug-resin complex granules (Example-10) whose particle size in between 125 μm-250 Maltodextrin, crystalline maltitol and Sucralose through #30 sieve (ASTM).
Step-02: Add Mono and Diglycerides, Acetylated Monoglyceride and coconut to the Groen Kettle; allow the mixture to melt.
Step-03: Add Maltitol Syrup 80/55 and Molten mixture from step-02 to BPG mixer. Mix in forward direction.
Step-04: Add sifted material from step-01 to the BPG mixer at temperature of approximately 105° F.-140° F. Close the lid. Mix in forward direction.
Step-05: Add Orange Flavor (Both liquid and powder) into BPG mixer. Mix in forward direction.
Step-06: Unload the material from BPG mixer and fill into plastic totes as per required dose. These soft chews tested in five volunteers no bitter taste observed during and after chewing.

### Example-23

### Dextromethorphan HBr 30 mg/5 ml Extended Release Suspension

| Material Name | mg/5 mL |
|---|---|
| Polymer encapsulated drug-resin complex | 100.80 |
| Maltitol Syrup 80/55 | 1.309 |
| Pharmaceutical Grade SUGAR | 0.483 |
| Propyl paraben Sodium USP | 0.001 |
| Methyl paraben Sodium USP | 0.006 |
| Xanthan gum USP/NF | 0.038 |
| Polysorbate 80 NF | 0.001 |
| Propylene Glycol USP/NF | 0.077 |
| Natural orange Flavor | 0.008 |
| FD&C Yellow #6/Sunset Yellow FCF AL | 0.0004 |
| Citric acid Anhydrous | 0.017 |
| Purified water USP | q.s. |

### Process:

Step 1: Maltitol Syrup 80/55 and Pharmaceutical grade sugar were dissolved in predefined amount of water under continuous stirring using a lab stirrer maintaining an RPM of 500-600.
Step 2: Methyl Paraben Sodium followed by Propyl Paraben Sodium (both previously sieved through #40 sieve) was dissolved in a predefined amount of water under continuous stirring. This solution was added to above solution.
Step 3: Xanthan gum was dispersed in predefined amount of water and kept the dispersion in refrigerator at 5° C. for 3-4 hrs.
Step 4: After that, Polysorbate 80K was added to the above xanthan gum dispersion under continuous stirring maintained a RPM of 500-700.
Step 5: step-04 dispersion was added to step-02 material under continuous stirring.
Step 6: Flavor followed by color was suspended in predefined amount of water and added to Step-02 suspension under continuous stirring. To this propylene glycol was added.
Step 7: pH of above Base suspension was adjusted to 3-3.5 pH using 5-10% citric acid solution. The suspension was made up to the volume using the remaining water.
Step 8: Encapsulated Dextromethorphan HBr was added to pH adjusted Base suspension under continuous stirring. Stirring continued till homogeneous suspension observed.
pH of suspension after addition of encapsulated active was about 3-3.5.
Up to 1 hr. sedimentation of suspension was not observed.
Taste and odor of suspension was acceptable and no after taste was observed.
In-Vitro Drug Release (Cumulative Percent Drug Release) of the Dextromethorphan HBr from the Extended Release Suspension in 0.1 N HCl.

Drug release studies were carried out using USP-27 dissolution apparatus (Type II, paddle). The formulated tablet was added to 500 ml of 0.1 N HCl. The temperature of the medium was maintained at 37° C.±0.5° C. and was stirred at 50 rpm. Sample (10 ml) withdrawn at predetermined time interval over a period of 12 hrs. After each sampling, equal amount of the medium was added. The sample withdrawn was filtered to remove particulate matter and drug content was analyzed by HPLC system at a wavelength of 280 nm.

| | Cumulative percent drug release |
|---|---|
| Time (hrs) | (Example-23) |
| 0.5 | 23.25 |
| 1 | 26.77 |
| 2 | 36.99 |
| 4 | 43.45 |
| 6 | 48.65 |
| 8 | 53.65 |
| 10 | 54.66 |
| 12 | 55.99 |

### Example 24

### Brompheniramine Maleate/Sodium Polystyrene Sulfonate Resin Complex (Ratio of Drug and Resin 40:55)

| Material Name | Amount (g) |
|---|---|
| Brompheniramine Maleate USP | 40.00 |
| Sodium polystyrene sulfonate USP/NF | 55.00 |
| Sucralose Powder NF | 5.00 |
| Purified water | q.s. |
| Total | 100.00 |

### Process:

The bromphenaramine maleate-sodium polystyrene sulfonate complex is prepared by, sifted the 55 Gyms. of sodium polystyrene sulfonate particle size in between 106-125 micrometers (μm) and 5 Gyms. of #30 (ASTM) sieved sucralose powder were dry mixed using rapid mixer granulator with an impellor RPM of 250 about 10 mins. then granulated by adding drug solution using rapid mixer granulator with an impellor RPM of 250 and chopper RPM of 750. Drug solution addition rate was 10 ml/minute. Then the granulated drug-resin complex was dried in an oven at 50° C.-505° C. till the moisture reaches to 5-7%. Moisture content was analyzed using MB45 moisture analyzer. Sift the dried drug-resin complex and the final particle size should be in between 106 μm-170 μm.

### Example 25

### Brompheniramine Maleate/Sodium polystyrene sulfonate Resin Complex (Ratio of Drug and Resin 30:65)

| Material Name | Amount (g) |
|---|---|
| Brompheniramine Maleate USP | 30.00 |
| Sodium polystyrene sulfonate USP/NF | 65.00 |
| Sucralose Powder NF | 5.00 |
| Purified water | q.s. |
| Total | 100.00 |

### Process:

The bromphenaramine maleate-sodium polystyrene sulfonate complex is prepared by, sifted the 65 Gyms. of sodium polystyrene sulfonate particle size in between 106-125 micrometers (μm) and 5 Gyms. of #30 (ASTM) sieved sucralose powder were dry mixed using rapid mixer granulator with an impellor RPM of 250 about 10 mins. then granulated by adding drug solution using rapid mixer granulator with an impellor RPM of 250 and chopper RPM of 750. Drug solution addition rate was 10 ml/minute. Then the granulated drug-resin complex was dried in an oven at 50° C.-505° C. till the moisture reaches to 5-7%. Moisture content was analyzed using MB45 moisture analyzer. Sift the dried drug-resin complex and the final particle size should be in between 106 μm-170 μm.

### Example 26

### Rapid Melt Controlled Release Tablets Combination of Polymer Coated Dextromethorphan

The following formula for controlled release rapid melt tablets containing 30 mg of Dextromethorphan HBr and 2 mg Bromphenaramine Maleate in each tablet. Coated Dextromethorphan-resin complex particles are from example-10, whose particle size should be 125 μm-250 μm and Brompheniramine Maleate-resin complex particles are from example-25, whose particle size should be 106 μm-170 μm.

| Material Name | mg/tablet |
|---|---|
| Polymer encapsulated | 100.78 |
| Dextromethorphan-resin complex | |
| Bromphenaramine Maleate-resin complex | 6.67 |
| Mannitol Granules USP/NF | 322.29 |
| Mannitol powder USP | 67.50 |
| Microcrystalline-Cellulose Powder | 135.00 |
| Sucralose powder NF | 3.38 |
| Natural orange Flavor | 3.38 |
| Crospovidone USP | 27.00 |
| Polysorbate 80 NF | 0.68 |
| FD&C Yellow #6 | 1.69 |
| Magnesium Stearate NF | 5.20 |
| Silicon Dioxide NF | 0.88 |
| Purified talc USP | 0.47 |
| Total | 675.00 mg/tablet |

### Process:

Step 1: Sift the Coated Dextromethorphan-resin complex particles are from example-10, whose particle size should be 125 μm-250 μm and Brompheniramine Maleate-resin complex particles are from example-25, whose particle size should be 106 μm-170 μm. through #40 sieve.
Step 2: Add #30 sieved ¾ parts of Mannitol granules, microcrystalline cellulose, and Mannitol powder to active.

### Step 3: Adsorb the Polysorbate 80 on Crospovidone.

Step 4: Mix remaining ¼ parts of Mannitol granules, sucralose, orange flavor and step-3 mixture then sieved through #40 sieve.
Step 5: Mix Step 2 and step 4 mixtures and sieved through #40 sieve.
Step 6: Add #50 sieved FD&C yellow #6 to step 5 mixtures and blended for 10 mins. using double cone blender at an RPM of 6-8.
Step 7: Sift Magnesium Stearate, talc and silicon dioxide through #50 sieve into a plastic container lined with poly bag, add to step 6 blended mixture and blend for 3-4 min using double cone blender at an RPM of 6-8 then compress blend part into tablet using ten stations pilot press tableting machine at an average hardness of 6 Kg.
These compressed tablets disintegrated in 25-30 seconds as tested in five volunteers with out observed bitter taste during disintegrate and after swallowed.

### Example 27

### Chlorpheniramine/Polacriline potassium Resin Complex

| Material Name | Amount (g) |
|---|---|
| Chlorpheniramine Maleate USP | 50.00 |
| Polacriline potassium resin USP/NF | 48.00 |
| Sucralose Powder NF | 2.00 |
| Purified water | q.s. |
| Total | 100.00 |

### Process:

The chlorpheniramine maleate-polacriline potassium resin complex is prepared by, sifted the 48 gyms. of Polacriline potassium resin particle size in between 106-125 micrometers (μm) and 2 Gyms. of #30 (ASTM) sieved sucralose powder were dry mixed using rapid mixer granulator with an impellor RPM of 250 about 10 mins. then granulated by adding drug solution using rapid mixer granulator with an impellor RPM of 250 and chopper RPM of 750. Drug solution addition rate was 10 ml/minute. Then the granulated drug-resin complex was dried in an oven at 50° C.-505° C. till the moisture reaches to 5-7%. Moisture content was analyzed using MB45 moisture analyzer. Sift the dried drug-resin complex and the final particle size should be in between 106 μm-170 μm.

### Example 28

### Chlorpheniramine/Polacriline potassium Resin Complex

| Material Name | Amount (g) |
|---|---|
| Chlorpheniramine Maleate USP | 40.00 |
| Polacriline potassium resin USP/NF | 68.00 |
| Sucralose Powder NF | 2.00 |
| Purified water | q.s. |
| Total | 100.00 |

### Process:

The chlorpheniramine maleate-Polacriline potassium resin complex is prepared by, sifted the 68 Gyms. of Polacriline potassium resin particle size in between 106-125 micrometers (μm) and 2 Gyms. of #30 (ASTM) sieved sucralose powder were dry mixed using rapid mixer granulator with an impellor RPM of 250 about 10 mins. then granulated by adding drug solution using rapid mixer granulator with an impellor RPM of 250 and chopper RPM of 750. Drug solution addition rate was 10 ml/minute. Then the granulated drug-resin complex was dried in an oven at 50° C.-505° C. till the moisture reaches to 5-7%. Moisture content was analyzed using MB45 moisture analyzer. Sift the dried drug-resin complex and the final particle size should be in between 106 μm-170 μm.

### Example-29

### Rapid Melt Controlled Release Tablets Combination of Polymer Coated Dextromethorphan Resin Complex and Chlorpheniramine Maleate/Resin Complex

The following formula is for controlled release rapid melt tablets containing 30 mg of dextromethorphan HBr and 2 mg chlorpheniramine maleate in each tablet. Coated dextromethorphan-resin complex particles are from example-10, whose particle size should be 125 μm-250 μm and Chlorpheniramine-resin complex particles are from example-28, whose particle size should be 106 μm-170 μm.

| Material Name | mg/tablet |
|---|---|
| Polymer encapsulated | 100.78 |
| Dextromethorphan-resin complex | |
| Chlorpheniramine Maleate-resin complex | 6.67 |
| Mannitol Granules USP/NF | 322.29 |
| Mannitol powder USP | 67.50 |
| Microcrystalline-Cellulose Powder NF/Ph. Eur. | 135.00 |
| Sucralose powder NF | 3.38 |
| Natural orange Flavor | 3.38 |
| Sodium starch glycolate NF | 27.00 |
| Sodium Lauryl sulfate NF | 0.68 |
| FD&C Yellow #6 | 1.69 |
| Stearic acid NF | 5.20 |
| Total | 675.00 mg/tablet |

### Process:

Step 1: Sift the Coated Dextromethorphan-resin complex particles are from example-10, whose particle size should be 125 μm-250 μm and Chlorpheniramine Maleate-resin complex particles are from example-25, whose particle size should be 106 μm-170 μm. through #40 sieve.
Step 2: Add #30 sieved ¾ parts of mannitol granules, microcrystalline cellulose, and Mannitol powder to active.
Step 3: Adsorb the Polysorbate 80 on sodium starch glycolate.
Step 4: Mix remaining ¼ parts of Mannitol granules, sucralose, orange flavor and step-3 mixture then sieved through #40 sieve.
Step 5: Mix Step 2 and step 4 mixtures and sieved through #40 sieve.
Step 6: Add #50 sieved FD&C yellow #6 to step 5 mixtures and blended for 10 mins. using double cone blender at an RPM of 6-8.
Step 7: Sift stearic acid through #50 sieve into a plastic container lined with poly bag, add to step 6 blended mixture and blend for 3-4 min using double cone blender at an RPM of 6-8 then compress blend part into tablet using ten stations pilot press tableting machine at an average hardness of 6 Kg.

These compressed tablets disintegrated in 25-30 seconds as tested in five volunteers with out observed bitter taste during disintegrate and after being swallowed.

What is claimed is:

1. A rapid melt composition for the relief of coughs and chest congestion comprising one or more bitter-tasting actives that is incorporated within a drug-resin ion-exchange complex micro-encapsulated within a carrier composition selected from the group consisting of polymers, biopolymers, fats, waxes, gums and mixtures thereof which is further incorporated within a fast dissolving polymer matrix selected from the group consisting of cellulose and cellulose derivatives, thermoplastic polymers, hydrogels, gums and mixtures thereof.

2. The rapid melt composition of claim 1 wherein said active is selected from the group consisting of antitussives, expectorants, antihistamines, decongestants and mixtures thereof.

3. The rapid melt composition of claim 2 wherein said active is selected from the group consisting of dextromethorphan, diclofenac, ibuprofen, phenylephrine, doxylamine, pseudoephedrine, imenhydrinate, brompheniramine, chlorpheniramine, brompheniramine maleate, chlorpheniramine maleate, phenylepherine hydrochloride, pseudoephedrine, their salts and mixtures thereof.

4. The rapid melt composition of claim 3 said active is encapsulated within a drug-resin complex wherein the resin is selected from the group consisting of acidic cation exchange resins with sulfonic acid functionality, weakly acidic cation exchange resins with carboloxylic functionality, strongly basic anion exchange resins with quaternary ammonium functionality, weakly basic anion exchange resins with secondary and tertiary amine functionality, polyamine methylene resins and mixtures thereof.

5. The rapid melt composition of claim 4 wherein said carrier is a water-swellable, hydrophilic polymer or water insoluble polymer selected from the group consisting of polyvinylpyrrolidine, (PVP) hydroxypropylcellulose (HPC), hydroxylpropylmethyl cellulose (HPMC), methyl cellulose, hydroxyl-ethylcellulose vinyl acetate copolymers, polysaccharides, polyethylene-oxide, methacrylic acid copolymers, maleic anhydride/methyl vinyl ether copolymers, natural gums or pharmaceutically acceptable hydrophobic polymers.

6. The rapid melt composition of claim 5 wherein said hydrophobic polymer is selected from the group consisting of ethyl cellulose, glyceryl behenate and glyceryl palmityl behenate.

7. The rapid melt composition of claim 6 wherein said anti-tussive is administered in an amount of from about 30 mg.-to about 60 mg per 500 mg of the total weight of the composition.

8. The composition of claim 7 wherein the composition is formulated as a rapidly dissolvable heterogeneous matrix, a flash-bead, gel or a compressed tablet.

9. The composition of claim 8 further comprising an excipient/diluent/bulking agent material selected from the group consisting of mannitol, dextrate, sorbitol, glycerol and mixtures thereof.

10. The composition of claim 9 further comprising a salivating agent.

11. The composition of claim 10, wherein the salivating agent is an emulsifier.

12. The composition of claim 11, wherein the emulsifier is sodium lauryl sulfate.

13. The composition of claim 12 wherein the emulsifier is polysorbate 80.

14. The composition of claim 13, wherein the excipient/diluent/bulking agent material comprises, silicon dioxide, sugars, starches, lactose, sucrose, sorbitol, fructose, talc, stearic acid, magnesium stearate, dicalcium phosphate, erythitol, xylitol, mannitol, maltitol, isomalt, dextrose, maltose, lactose, microcrystalline celluloses and mixtures thereof.

15. The composition of claim 14 further comprising a high potency sweetener.

16. The composition of claim 15 further comprising a mineral salt.

17. The rapid melt composition of claim 2 wherein said active is selected from the group consisting of dextromethorphan hydrobromide.

18. The rapid melt composition of claim 5 wherein said active is selected from the group consisting of dextromethorphan hydrobromide.

19. The rapid melt composition of claim 18 formulated without a binder.

20. The rapid melt composition of claim 18 further comprising a tablet lubricating agent selected from the group consisting of silicon dioxide, stearic acid, magnesium stearate and mixtures thereof.
